(19) United States
(12) Patent Application Publication
(10) Pub. No.: US 2021/0196780 A1
(43) Pub. Date: Jul. 1, 2021

(54) INFLAMMATORY CYTOKINE PRODUCTION INHIBITOR

(71) Applicant: SAISEI PHARMA CO., LTD., Osaka (JP)

(72) Inventor: Toshio Inui, Osaka (JP)

(73) Assignee: SAISEI PHARMA CO., LTD., Osaka (JP)

(21) Appl. No.: 17/260,891

(22) PCT Filed: Jul. 16, 2019
(86) PCT No.: PCT/JP2019/027870

§ 371 Date: Mar. 3, 2021

(30) Foreign Application Priority Data
Jul. 17, 2018 (JP) 2018-133954

(51) Int. Cl. A61K 36/81

(52) U.S. Cl. CPC A61K 36/81

(57) ABSTRACT

Inflammatory cytokine production inhibitors containing a tomato extract as an active ingredient, which inhibit the production of the inflammatory cytokines TNF-α and IL-6 and can be applied to drugs effective against inflammatory diseases caused by overproduction of these inflammatory cytokines, such as rheumatoid arthritis, ulcerative colitis, Crohn's disease, and type 2 diabetes, as well as to foods and drinks such as health foods, wherein the tomato extract is a raw tomato extract that is a high-molecular-weight fraction having a molecular weight of 10,000 or higher obtained by molecular weight fractionation.

### TECHNICAL FIELD

The present invention relates to inflammatory cytokine production inhibitors containing a tomato extract as an active ingredient, and inflammatory cytokine production inhibitory foods and drinks.

### BACKGROUND ART

Inflammatory cytokines are substances which are produced from lymphocytes, macrophages, and other cells and which are involved in inflammatory responses associated with bacterial or viral infection, tumors, or histological damage. For example, inflammatory cytokines such as interleukin-1 (IL-1), interleukin-6 (IL-6), and tumor necrosis factor (TNF-α) originally have purposeful functions such as activation of immune functions against invasion by pathogenic bacteria, but continuous overproduction thereof due to certain causes is known to induce a variety of diseases such as rheumatoid arthritis, ulcerative colitis, Crohn's disease, type 2 diabetes, and obesity (especially insulin resistance).

From the standpoint of neuroinflammation, they are also known to induce depression, for example.

In this context, studies have been performed for development of drugs for inhibiting the production of the inflammatory cytokines such as TNF-α in the pathological conditions mentioned above. For example, in addition to the conventional anti-inflammatory agents such as ibuprofen and indomethacin, a variety of chemical substances have been proposed (for example, see Patent Literatures 1 to 3). However, the aforementioned diseases often follow a chronic course and may require prolonged treatment. Thus, it is particularly desirable to provide a safe compound without side effects. In such a situation, effective compounds have not yet appeared.

From the aforementioned standpoint, some studies (Patent Literatures 4 and 5) have proposed inflammatory cytokine production inhibitors which contain juice and/or extract of fruit, or the iron-binding glycoprotein lactoferrin, rather than organic compounds, as an active ingredient mainly for the purpose of ensuring safety.

To search inflammatory cytokine production inhibitors which are more effective and are safe even when orally administered, the present inventors analyzed the effects of extracts of vegetables taken daily and safely. The present inventors then found that an extracted component of tomatoes has an excellent effect of inhibiting the production of inflammatory cytokines and confirmed that this tomato extract significantly inhibits production of inflammatory cytokines, particularly IL-6 and TNF-α. This has led to the completion of the present invention.

Meanwhile, the use of a tomato extract as an anti-allergic agent has been proposed before (Patent Literature 6).

The anti-allergic agent proposed in this patent literature is due to the effect of inhibiting release of chemical mediators such as histamine and leukotriene from mast cells according to the actual pathogenic mechanism of allergic diseases, and does not aim to inhibit the production of inflammatory cytokines as in the present invention.

From this standpoint, tomato extracts are expected to have a variety of pharmacological actions. The confirmation of the effect of inhibiting the production of inflammatory cytokines as analyzed by the present inventors is also considered as a very specific and effective use of tomato extracts.

### CITATION LIST

### Patent Literature

Patent Literature 1: JP 2015-174850 A

Patent Literature 2: JP 2014-101329 A

Patent Literature 3: JP 2009-013106 A

Patent Literature 4: JP 2005-089304 A

Patent Literature 5: JP 2006-069995 A

Patent Literature 6: JP 2002-080387 A

### SUMMARY OF INVENTION

### Technical Problem

Accordingly, the present invention aims to provide inflammatory cytokine production inhibitors which inhibit the production of the inflammatory cytokines TNF-α and IL-6 and can be applied to drugs effective against inflammatory diseases caused by overproduction of these inflammatory cytokines, such as rheumatoid arthritis, ulcerative colitis, Crohn's disease, and type 2 diabetes, as well as to foods and drinks such as health foods.

### Solution to Problem

To solve the above issue, the present invention may be provided as any of the following basic embodiments:

(1) an inflammatory cytokine production inhibitor, containing a tomato extract as an active ingredient;

(2) the inflammatory cytokine production inhibitor according to the embodiment (1), wherein the inflammatory cytokine is interleukin-1 (IL-1), interleukin-6 (IL-6), or tumor necrosis factor (TNF-α);

(3) the inflammatory cytokine production inhibitor according to the embodiment (1) or (2), wherein the tomato extract is a fraction having a molecular weight of 10,000 or higher obtained by molecular weight fractionation of squeezed juice of raw tomatoes;

(4) a drug or a food or drink, containing the inflammatory cytokine production inhibitor according to the embodiment (1) to (3); and

(5) the drug or the food or drink according to the embodiment (4) in an orally administrable or orally ingestible form.

### Advantageous Effects of Invention

The present invention provides inflammatory cytokine production inhibitors which are highly safe via oral administration or ingestion.

The inflammatory cytokine production inhibitors provided by the present invention inhibit overproduction of inflammatory cytokines such as interleukin-1 (IL-1), interleukin-6 (IL-6), and tumor necrosis factor (TNF-α), for example. Thus, these inhibitors may serve as therapeutic agents effective against a variety of diseases caused by overproduction of these cytokines, such as rheumatoid arthritis, ulcerative colitis, and Crohn's disease, as well as type 2 diabetes, depression, and obesity. Further, these inhibitors have the advantage of providing daily healthcare effectively by daily oral ingestion.

### BRIEF DESCRIPTION OF DRAWINGS

FIG. 1 is a graph showing the results after three hours of Experimental Example 1.

FIG. 2 is a graph showing the results after six hours of Experimental Example 1.

FIG. 3 is a graph showing the results after three hours of Experimental Example 2.

FIG. 4 is a graph showing the results after six hours of Experimental Example 2.

FIG. 5 is a graph showing the results after three hours of Experimental Example 3.

FIG. 6 is a graph showing the results after six hours of Experimental Example 3.

FIG. 7 is a graph showing the results after three hours of Experimental Example 4 as a comparative example.

FIG. 8 is a graph showing the results after three hours of Experimental Example 5.

FIG. 9 is a graph showing the results after six hours of Experimental Example 5.

FIG. 10 is a graph showing the results after three hours of Experimental Example 6.

FIG. 11 is a graph showing the results after six hours of Experimental Example 6.

FIG. 12 is a graph showing the results after three hours of Experimental Example 7.

FIG. 13 is a graph showing the results after six hours of Experimental Example 7.

### DESCRIPTION OF EMBODIMENTS

As described above, the basic embodiments of the present invention include inflammatory cytokine production inhibitors containing a tomato extract as an active ingredient.

The tomato extract, which is an active ingredient in the inflammatory cytokine production inhibitors provided by the present invention, is an extract obtained by homogenizing ripe raw tomatoes and removing solids, i.e., obtained from squeezed juice.

The tomato material may be of any cultivar. Any cultivar eaten raw or processed that is commonly available in the market may be used.

In the preparation of squeezed juice from dried tomatoes, they are usually homogenized together with an appropriate buffer solution such as purified water or saline. In contrast, in the present invention in which ripe raw tomatoes having high water content are used, the raw tomatoes themselves may be directly crushed without using a buffer solution, and solids may then be removed by a usual method such as centrifugation to obtain squeezed juice.

Then, a tomato extract may be obtained from this squeezed juice. In the present invention, the extraction may preferably be carried out using a centrifugal ultrafilter to obtain the extract as a fraction having a molecular weight of 10,000 or higher.

Since squeezed juice of raw tomatoes contains a large amount of saccharides, the extract is preferably adjusted to 10,000 or higher on a molecular weight basis.

Examples of the centrifugal ultrafilter used include a variety of filters that may be suitable for the target molecular weight fractionation, such as Amicon Ultra® series available from Merck Millipore.

Thus, an extract having a molecular weight of 10,000 or higher can be obtained as a raw tomato extract. The resulting extract may be used as it is. Alternatively, it may be prepared into a concentrated liquid by solvent evaporation or prepared into a dried product before use.

The drying may be carried out by a usual drying technique such as vacuum drying, freeze drying, or spray drying. Among these, freeze drying is preferably performed to form a dried product.

The raw tomato extract provided by the present invention has an excellent effect in inhibiting the production of inflammatory cytokines such as IL-1, IL-6, and TNF-α, and is useful as an inflammatory cytokine production inhibitor.

The inflammatory cytokine production inhibitors of the present invention may be administered in any manner in accordance with the purpose of administration, the type of disease, and the particular symptoms. The inhibitors may be in the dosage form of a tablet, a capsule, granules, powder, a powdered medicine, liquid, or other forms for direct administration or may be mixed into a food or drinking water for administration, and are desirably orally administered.

These dosage forms can be prepared by usual conventional methods. Appropriate additives such as dextrin, lactose, cornstarch, emulsifiers, antiseptics, vehicles, expanders, sweetening agents, flavors, and colorants may be incorporated as long as the advantageous effect of the present invention is not impaired.

The inflammatory cytokine production inhibitors of the present invention may also be used as foods or drinks. Examples of such foods or drinks include non-alcoholic drinks such as soft drinks and juices, alcoholic drinks, fermented drinks such as yogurt, hard sweets such as tablets and candies, chewable sweets such as gum and gummies, nutritional supplements containing, for example, vitamins, minerals, amino acids, or proteins, and other forms.

Examples of such foods or drinks functionally include foods for specified health uses (“TOKUHO”), foods with nutrient function claims, and foods with function claims.

The foregoing agents and foods and drinks have an ability to inhibit the production of inflammatory cytokines and can be very useful for the prevention, treatment, improvement or relapse prevention of a variety of pathological conditions induced by overproduction of inflammatory cytokines.

For the inflammatory cytokine production inhibitors of the present invention, the dose for achieving the ability to inhibit the production of inflammatory cytokines is not limited, and may vary in accordance with the purpose of administration, the type of disease, and the particular symptoms. For example, the amount may be adjusted so that 1 to 5000 mg, preferably 5 to 1000 mg, more preferably 10 to 200 mg, of the raw tomato extract can be ingested per day.

The raw tomato extract according to the present invention is obtained from squeezed juice of ripe tomatoes which are eaten daily, and thus is highly safe and causes no toxicity problem at the doses used in the present invention.

### EXAMPLES

The present invention is described in more detail below with reference to examples and experimental examples. These are mere examples and are not at all intended to limit the present invention.

### Example 1: Preparation of Raw Tomato Extract

An amount of 103.96 g of ripe raw tomatoes (grown in Kagoshima Prefecture) were cut and crushed in a mortar. The resulting material was centrifuged at 4000×g for 15 minutes and 27.5 mL of the liquid portion was collected.

A 20-mL portion of the liquid portion was subjected to centrifugal ultrafiltration at 4000×g using the centrifugal ultrafilter Amicon Ultra-15 (MWCO: 10,000).

After the centrifugation, a high-molecular-weight fraction having a molecular weight of 10,000 or higher and a low-molecular-weight fraction having a molecular weight of 10,000 or lower were taken and freeze-dried to obtain raw tomato extracts.

The raw tomato extract prepared as above mainly contains saccharides with some proteins and can be directly used as an inflammatory cytokine production inhibitor of the present invention.

The raw tomato extract according to the present invention corresponds to the molecular weight fraction having a molecular weight of 10,000 or higher prepared as above, but for the purpose of comparison, the prepared molecular weight fraction having a molecular weight of 10,000 or lower was also subjected to the following experiments.

In the following experiments, the prepared raw tomato extracts were each adjusted to a concentration of 100 μg/mL in a DMEN medium (Dulbecco's modified Eagle's medium+10% bovine serum+0.1% antibiotic/antimicrobial) before application to cells.

### Experimental Example 1: Effect of Raw Tomato Extract on TNF-α mRNA Expression in Mouse Microglial Cells (MG6) in Presence of LPS

The following describes a specific procedure.

(1) A 6-well plate was seeded with a mouse microglial cell line (MG6) at 1×10³ cells/mL.

(2) After three to four days, solutions of four groups, i.e., control (DMEN medium, 1 mL/well), LPS (5 ng/mL), LPS (5 ng)+high-molecular-weight raw tomato extract (100 μg) (liquid volume: 1 mL), and high-molecular-weight raw tomato extract (100 μg/mL) were applied to the cells.

(3) After three hours and after six hours, the culture medium was removed and 0.3 mL of an RLT solution (RNeasy Mini kit, QIAGEN) (containing 1% R-mercaptoethanol) was added to each well.

(4) The cells were scraped using a cell scraper.

(5) The cell suspension was mixed with 0.3 mL of 70% ethanol.

(6) The above solution was put into a spin column (RNeasy Mini kit, QIAGEN) and centrifuged at 8000×g for one minute.

(7) To the spin column was added 700 μL of an RW1 solution (RNeasy Mini kit, QIAGEN), followed by centrifugation at 8000×g for one minute.

(8) To the spin column was added 500 μL of an RPE solution (RNeasy Mini kit, QIAGEN), followed by centrifugation at 8000×g for one minute. This step was repeated twice.

(9) The spin column was placed in a new tube (1.5 mL) and 50 μL of RNase-free water was added. The mixture was left to stand for one minute, and then centrifuged at 12000×g for one minute. This step was repeated twice.

(10) A 11-μL portion of this aqueous solution was used with Versco cDNA Synthesis Kit (Thermoscientific) to prepare cDNA.

(11) The resulting cDNA solution was diluted 10-fold with RNase-free water.

(12) This diluted cDNA solution was used to determine the expression of GAPDH mRNA and TNF-α mRNA by standard real-time RT-PCR.

(13) The PCR was performed using a 96-well plate.

(14) Each well was charged with 8.5 μL of water, 2.5 μL of cDNA sample solution, 1.5 μL of GAPDH mRNA detection primers or TNF-α mRNA detection primers (forward+reverse), and 12.5 μL of Syber green mater mix.

(15) The thermal cycle conditions for PCR were as follows: Hold, 94° C., 10 min+3 step PCR (40 cycles; 94° C., 30 min+55° C., 30 sec+72° C., 30 sec)+Hold, 72° C., 1 min.

(16) For each sample of each group, the TNF-α mRNA/GAPDH mRNA value was determined and converted into percentage with the average value of the control taken as 100%.

(17) A statistical significance test was performed by analysis of variance followed by the Tukey-Kramer method.

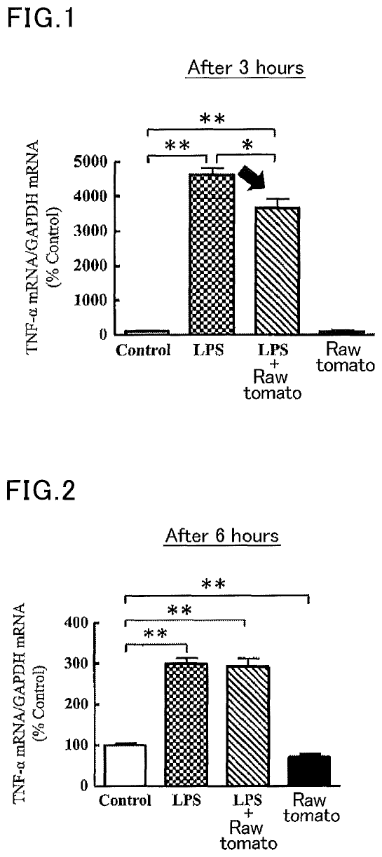

The results are shown in FIGS. 1 and 2.

FIG. 1 shows the results after three hours and FIG. 2 shows the results after six hours.

As demonstrated from the results shown in FIG. 1, the raw tomato extract according to the present invention is understood to effectively inhibit the expression of TNF-α mRNA in the results after three hours.

### Experimental Example 2: Effect of Raw Tomato Extract on IL-1β mRNA Expression in Mouse Microglial Cells (MG6) in Presence of LPS

The following describes a specific procedure, which follows Experimental Example 1.

(1) A 6-well plate was seeded with a mouse microglial cell line (MG6) at 1×10⁵ cells/mL.

(2) After three to four days, solutions of four groups, i.e., control (DMEN medium, 1 mL/well), LPS (5 ng/mL), LPS (5 ng)+high-molecular-weight raw tomato extract (100 μg) (liquid volume: 1 mL), and high-molecular-weight raw tomato extract (100 μg/mL) were applied to the cells.

(3) After three hours and after six hours, the culture medium was removed and 0.3 mL of an RLT solution (RNeasy Mini kit, QIAGEN) (containing 1% R-mercaptoethanol) was added to each well.

(4) The cells were scraped using a cell scraper.

(5) The cell suspension was mixed with 0.3 mL of 70% ethanol.

(6) The above solution was put into a spin column (RNeasy Mini kit, QIAGEN) and centrifuged at 8000×g for one minute.

(7) To the spin column was added 700 μL of an RW1 solution (RNeasy Mini kit, QIAGEN), followed by centrifugation at 8000×g for one minute.

(8) To the spin column was added 500 μL of an RPE solution (RNeasy Mini kit, QIAGEN), followed by centrifugation at 8000×g for one minute. This step was repeated twice.

(9) The spin column was placed in a new tube (1.5 mL) and 50 μL of RNase-free water was added. The mixture was left to stand for one minute, and then centrifuged at 12000×g for one minute. This step was repeated twice.

(10) A 11-μL portion of this aqueous solution was used with Versco cDNA Synthesis Kit (Thermoscientific) to prepare cDNA.

(11) The resulting cDNA solution was diluted 10-fold with RNase-free water.

(12) This diluted cDNA solution was used to determine the expression of GAPDH mRNA and IL-1B mRNA by standard real-time RT-PCR.

(13) The PCR was performed using a 96-well plate.

(14) Each well was charged with 8.5 μL of water, 2.5 μL of cDNA sample solution, 1.5 μL of GAPDH mRNA detection primers or IL-1B mRNA detection primers (forward+reverse), and 12.5 μL of Syber green mater mix.

(15) The thermal cycle conditions for PCR were as follows: Hold, 94° C., 10 min+3 step PCR (40 cycles; 94° C., 30 min+55° C., 30 sec+72° C., 30 sec)+Hold, 72° C., 1 min.

(16) For each sample of each group, the IL-1B mRNA/GAPDH mRNA value was determined and converted into percentage with the average value of the control taken as 100%.

(17) A statistical significance test was performed by analysis of variance followed by the Tukey-Kramer method.

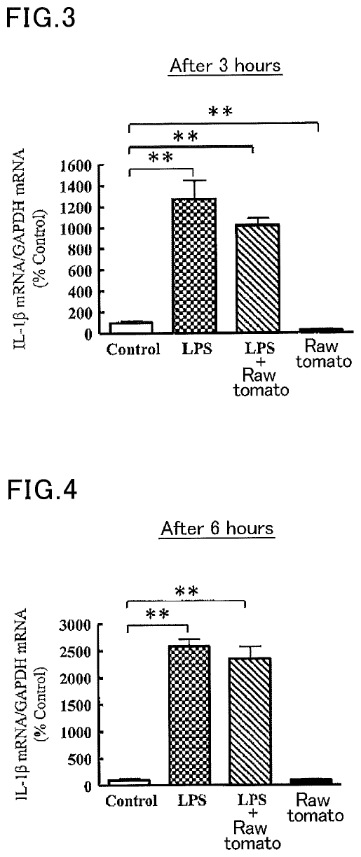

The results are shown in FIGS. 3 and 4.

FIG. 3 shows the results after three hours and FIG. 4 shows the results after six hours.

As demonstrated from the results shown in the figure, the raw tomato extract according to the present invention is understood to effectively inhibit the expression of IL-1β mRNA in the results after three hours.

### Experimental Example 3: Effect of Raw Tomato Extract on IL-6 mRNA Expression in Mouse Microglial Cells (MG6) in Presence of LPS

The following describes a specific procedure, which follows Experimental Example 1.

(1) A 6-well plate was seeded with a mouse microglial cell line (MG6) at 1×10³ cells/mL.

(2) After three to four days, solutions of four groups, i.e., control (DMEN medium, 1 mL/well), LPS (5 ng/mL), LPS (5 ng)+high-molecular-weight raw tomato extract (100 μg) (liquid volume: 1 mL), and high-molecular-weight raw tomato extract (100 μg/mL) were applied to the cells.

(3) After three hours and after six hours, the culture medium was removed and 0.3 mL of an RLT solution (RNeasy Mini kit, QIAGEN) (containing 1% β-mercaptoethanol) was added to each well.

(4) The cells were scraped using a cell scraper.

(5) The cell suspension was mixed with 0.3 mL of 70% ethanol.

(6) The above solution was put into a spin column (RNeasy Mini kit, QIAGEN) and centrifuged at 8000×g for one minute.

(7) To the spin column was added 700 μL of an RW1 solution (RNeasy Mini kit, QIAGEN), followed by centrifugation at 8000×g for one minute.

(8) To the spin column was added 500 μL of an RPE solution (RNeasy Mini kit, QIAGEN), followed by centrifugation at 8000×g for one minute. This step was repeated twice.

(9) The spin column was placed in a new tube (1.5 mL) and 50 μL of RNase-free water was added. The mixture was left to stand for one minute, and then centrifuged at 12000×g for one minute. This step was repeated twice.

(10) A 11-μL portion of this aqueous solution was used with Versco cDNA Synthesis Kit (Thermoscientific) to prepare cDNA.

(11) The resulting cDNA solution was diluted 10-fold with RNase-free water.

(12) This diluted cDNA solution was used to determine the expression of GAPDH mRNA and IL-6 mRNA by standard real-time RT-PCR.

(13) The PCR was performed using a 96-well plate.

(14) Each well was charged with 8.5 μL of water, 2.5 μL of cDNA sample solution, 1.5 μL of GAPDH mRNA detection primers or IL-6 mRNA detection primers (forward+reverse), and 12.5 μL of Syber green mater mix.

(15) The thermal cycle conditions for PCR were as follows: Hold, 94° C., 10 min+3 step PCR (40 cycles; 94° C., 30 min+55° C., 30 sec+72° C., 30 sec)+Hold, 72° C., 1 min.

(16) For each sample of each group, the IL-6 mRNA/GAPDH mRNA value was determined and converted into percentage with the average value of the control taken as 100%.

(17) A statistical significance test was performed by analysis of variance followed by the Tukey-Kramer method.

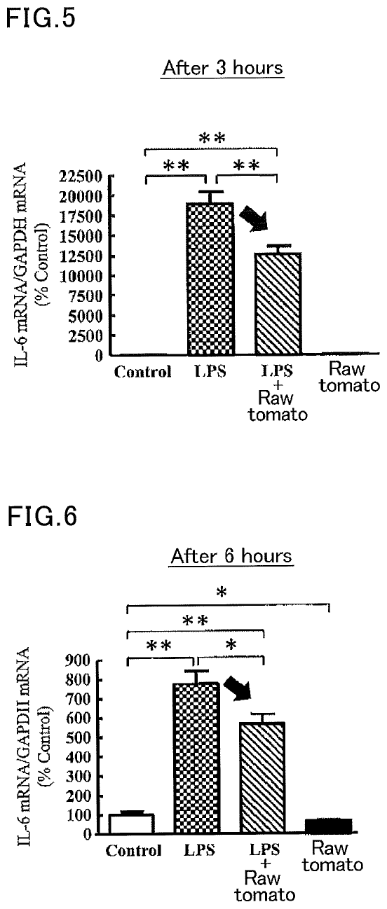

The results are shown in FIGS. 5 and 6.

FIG. 5 shows the results after three hours and FIG. 6 shows the results after six hours.

As demonstrated from the results shown in the figures, the raw tomato extract according to the present invention is understood to effectively inhibit the expression of IL-6 mRNA in the results after three hours and after six hours.

### Experimental Example 4: Effect of Raw Tomato Extract on TNF-α mRNA Expression in Mouse Microglial Cells (MG6) in Presence of LPS

For the purpose of comparison, the low-molecular-weight fraction (molecular weight: 10,000 or lower) as a raw tomato extract was analyzed for the effect of inhibiting the production of inflammatory cytokines.

The analysis was carried out by the same procedure as in Experimental Example 1, but using 100 μg/mL of low-molecular-weight raw tomato extract.

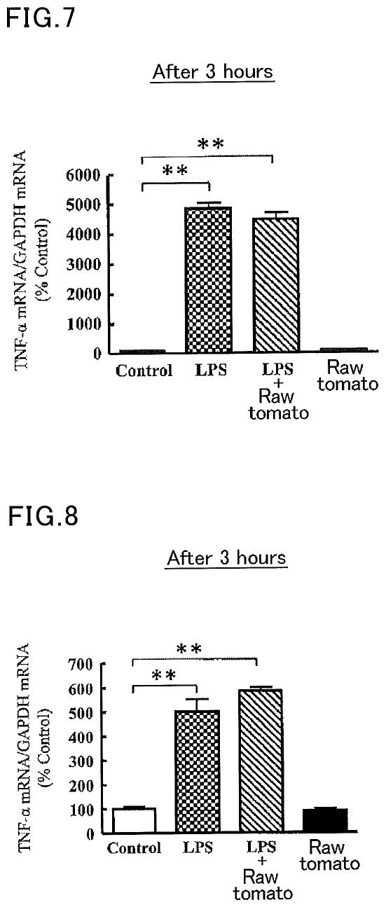

The results are shown in FIG. 7.

As demonstrated from the results shown in FIG. 7, the low-molecular-weight raw tomato extract is found to have no effect in inhibiting the production of inflammatory cytokines, while the high-molecular-weight fraction raw tomato extract having a molecular weight of 10,000 or higher is found to have an effect of inhibiting the production of inflammatory cytokines.

### Experimental Example 5: Effect of Raw Tomato Extract on TNF-α mRNA Expression in Mouse Macrophage (Raw264) in Presence of LPS

The analysis was carried out by the same procedure as in Experimental Example 1, but using a mouse macrophage cell line (Raw264) instead of the mouse microglial cell line (MG6).

Specifically, the analysis was carried out by the following specific procedure.

(1) A 6-well plate was seeded with a mouse macrophage cell line (Raw264) at 1×10³ cells/mL.

(2) After three to four days, solutions of four groups, i.e., control (DMEN medium, 1 mL/well), LPS (5 ng/mL), LPS (5 ng)+high-molecular-weight raw tomato extract (100 μg) (liquid volume: 1 mL), and high-molecular-weight raw tomato extract (100 μg/mL) were applied to the cells.

(3) After three hours and after six hours, the culture medium was removed and 0.3 mL of an RLT solution (RNeasy Mini kit, QIAGEN) (containing 1% β-mercaptoethanol) was added to each well.

(4) The cells were scraped using a cell scraper.

(5) The cell suspension was mixed with 0.3 mL of 70% ethanol.

(6) The above solution was put into a spin column (RNeasy Mini kit, QIAGEN) and centrifuged at 8000×g for one minute.

(7) To the spin column was added 700 μL of an RW1 solution (RNeasy Mini kit, QIAGEN), followed by centrifugation at 8000×g for one minute.

(8) To the spin column was added 500 μL of an RPE solution (RNeasy Mini kit, QIAGEN), followed by centrifugation at 8000×g for one minute. This step was repeated twice.

(9) The spin column was placed in a new tube (1.5 mL) and 50 μL of RNase-free water was added. The mixture was left to stand for one minute, and then centrifuged at 12000×g for one minute. This step was repeated twice.

(10) A 11 μL portion of this aqueous solution was used with Versco cDNA Synthesis Kit (Thermoscientific) to prepare cDNA.

(11) The resulting cDNA solution was diluted 10-fold with RNase-free water.

(12) This diluted cDNA solution was used to determine the expression of GAPDH mRNA and TNF-α mRNA by standard real-time RT-PCR.

(13) The PCR was performed using a 96-well plate.

(14) Each well was charged with 8.5 μL of water, 2.5 μL of cDNA sample solution, 1.5 μL of GAPDH mRNA detection primers or TNF-α mRNA detection primers (forward+reverse), and 12.5 μL of Syber green mater mix.

(15) The thermal cycle conditions for PCR were as follows: Hold, 94° C., 10 min+3 step PCR (40 cycles; 94° C., 30 min+55° C., 30 sec+72° C., 30 sec)+Hold, 72° C., 1 min.

(16) For each sample of each group, the TNF-α mRNA/GAPDH mRNA value was determined and converted into percentage with the average value of the control taken as 100%.

(17) A statistical significance test was performed by analysis of variance followed by the Tukey-Kramer method.

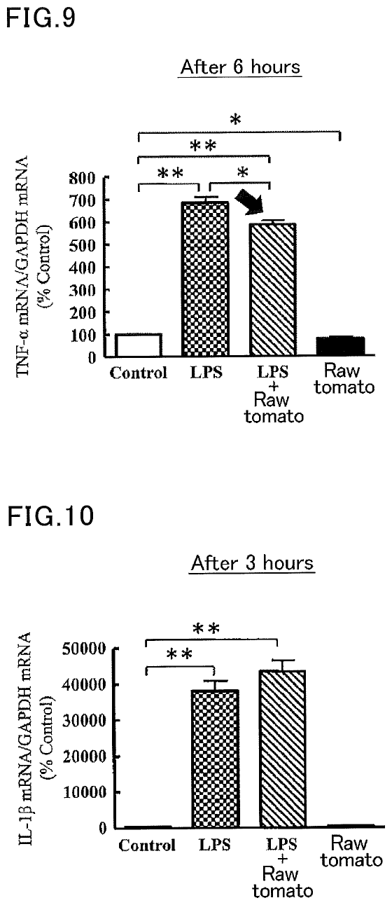

The results are shown in FIGS. 8 and 9.

FIG. 8 shows the results after three hours and FIG. 9 shows the results after six hours.

As demonstrated from the results shown in FIG. 9, the raw tomato extract according to the present invention is understood to effectively inhibit the expression of TNF-α mRNA in the results after six hours.

### Experimental Example 6: Effect of Raw Tomato Extract on IL-1β mRNA Expression in Mouse Macrophage (Raw264) in Presence of LPS

The analysis was performed in accordance with the procedure of Experimental Example 5.

Specifically,

(1) A 6-well plate was seeded with a mouse macrophage cell line (Raw264) at 1×10³ cells/mL.

(2) After three to four days, solutions of four groups, i.e., control (DMEN medium, 1 mL/well), LPS (5 ng/mL), LPS (5 ng)+high-molecular-weight raw tomato extract (100 μg) (liquid volume: 1 mL), and high-molecular-weight raw tomato extract (100 μg/mL) were applied to the cells.

(3) After three hours and after six hours, the culture medium was removed and 0.3 mL of an RLT solution (RNeasy Mini kit, QIAGEN) (containing 1% R-mercaptoethanol) was added to each well.

(4) The cells were scraped using a cell scraper.

(5) The cell suspension was mixed with 0.3 mL of 70% ethanol.

(6) The above solution was put into a spin column (RNeasy Mini kit, QIAGEN) and centrifuged at 8000×g for one minute.

(7) To the spin column was added 700 μL of an RW1 solution (RNeasy Mini kit, QIAGEN), followed by centrifugation at 8000×g for one minute.

(8) To the spin column was added 500 μL of an RPE solution (RNeasy Mini kit, QIAGEN), followed by centrifugation at 8000×g for one minute. This step was repeated twice.

(9) The spin column was placed in a new tube (1.5 mL) and 50 μL of RNase-free water was added. The mixture was left to stand for one minute, and then centrifuged at 12000×g for one minute. This step was repeated twice.

(10) A 11-μL portion of this aqueous solution was used with Versco cDNA Synthesis Kit (Thermoscientific) to prepare cDNA.

(11) The resulting cDNA solution was diluted 10-fold with RNase-free water.

(12) This diluted cDNA solution was used to determine the expression of GAPDH mRNA and IL-1B mRNA by standard real-time RT-PCR.

(13) The PCR was performed using a 96-well plate.

(14) Each well was charged with 8.5 μL of water, 2.5 μL of cDNA sample solution, 1.5 μL of GAPDH mRNA detection primers or IL-1β mRNA detection primers (forward+reverse), and 12.5 μL of Syber green mater mix.

(15) The thermal cycle conditions for PCR were as follows: Hold, 94° C., 10 min+3 step PCR (40 cycles; 94° C., 30 min+55° C., 30 sec+72° C., 30 sec)+Hold, 72° C., 1 min.

(16) For each sample of each group, the IL-1β mRNA/GAPDH mRNA value was determined and converted into percentage with the average value of the control taken as 100%.

(17) A statistical significance test was performed by analysis of variance followed by the Tukey-Kramer method.

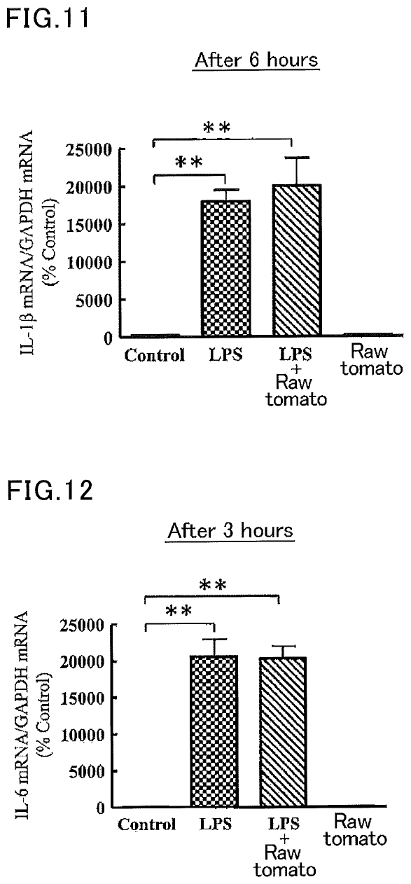

The results are shown in FIGS. 10 and 11.

FIG. 10 shows the results after three hours and FIG. 11 shows the results after six hours.

### Experimental Example 7: Effect of Raw Tomato Extract on IL-6 mRNA Expression in Mouse Macrophage (Raw264) in Presence of LPS

The effect on IL-6 mRNA expression was analyzed using a mouse macrophage cell line (Raw264) in accordance with Experimental Examples 5 and 6.

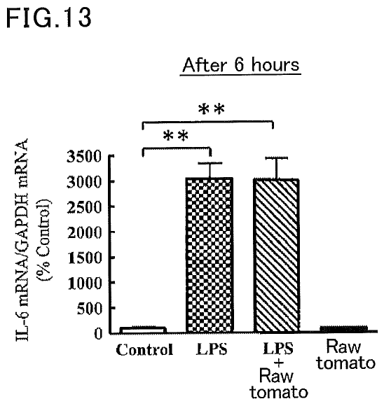

The results are shown in FIGS. 12 and 13.

FIG. 12 shows the results after three hours and FIG. 13 shows the results after six hours.

From the above analysis results in Experimental Examples 5 to 7, the raw tomato extract according to the present invention is understood to significantly inhibit an increase in TNF-α mRNA expression caused by LSP stimulation, particularly in the mouse macrophage cell line (Raw264).

As can be well understood from the results of the above experimental examples, the raw tomato extract according to the present invention, which is a high-molecular-weight fraction having a molecular weight of 10,000 or higher, better exhibits an effect of inhibiting the production of inflammatory cytokines than the low-molecular-weight fraction. Therefore, it is demonstrated that the high-molecular-weight tomato extract according to the present invention has an anti-inflammatory effect.

### INDUSTRIAL APPLICABILITY

The present invention provides inflammatory cytokine production inhibitors which are highly safe via oral administration or ingestion.

The inflammatory cytokine production inhibitors provided by the present invention inhibit overproduction of inflammatory cytokines such as interleukin-1 (IL-1), interleukin-6 (IL-6), and tumor necrosis factor (TNF-α), and thus may serve as therapeutic agents effective against a variety of diseases caused by overproduction of these cytokines, such as rheumatoid arthritis, ulcerative colitis, Crohn's disease, type 2 diabetes, obesity (especially insulin resistance), and depression. Further, these inhibitors have the advantage of providing daily healthcare effectively by daily oral ingestion. Accordingly, these inhibitors have significant industrial applicability.

1. An inflammatory cytokine production inhibitor, comprising a tomato extract as an active ingredient.

2. The inflammatory cytokine production inhibitor according to claim 1,
wherein the inflammatory cytokine is interleukin-1 (IL-1), interleukin-6 (IL-6), or tumor necrosis factor (TNF-α).

3. The inflammatory cytokine production inhibitor according to claim 1,
wherein the tomato extract is a fraction having a molecular weight of 10,000 or higher obtained by molecular weight fractionation of squeezed juice of raw tomatoes.

4. A drug or a food or drink, comprising the inflammatory cytokine production inhibitor according to claim 1.

5. The drug or the food or drink according to claim 4,
wherein the drug or the food or drink is in an orally administrable or orally ingestible form.
